(19) United States
(12) Patent Application Publication
(10) Pub. No.: US 2022/0117745 A1
(43) Pub. Date: Apr. 21, 2022

(54) Self-Broaching Neck Preserving Hip Stem

(71) Applicant: Howmedica Osteonics Corp., Mahwah, NJ (US)

(72) Inventors: Shammodip Roy, Wayne, NJ (US); Daniel Rudolf Scholl, Andover, NJ (US); Tyler Saunders, Park Ridge, NJ (US)

(73) Assignees: Howmedica Osteonics Corp., Mahwah, NJ (US); Howmedica Osteonics Corp., Mahwah, NJ (US)

(21) Appl. No.: 17/499,099

(22) Filed: Oct. 12, 2021

Related U.S. Application Data
(60) Provisional application No. 63/092,547, filed on Oct. 16, 2020.

(51) Int. Cl. A61F 2/36

(52) U.S. Cl. CPC A61F 2/3609; A61F 2/3662; A61F 2002/3625; A61F 2002/3652

(57) ABSTRACT

A self-broaching, neck-preserving hip stem includes a set of rasp teeth on a distal-lateral portion of the stem to allow the stem to be impacted into a femur without the use of surgical broaches. The rasp teeth are disposed on a relief surface. The stem includes a length, radius of curvature, and a dual-taper in the anterior-posterior and distal-lateral directions which allow the neck to self-broach and seat itself proximal to the conventional resection level to preserve greater bone stock in the femur.

### CROSS-REFERENCE TO RELATED APPLICATIONS

This application claims the benefit of the filing date of U.S. Provisional Application No. 63/092,547 filed Oct. 16, 2020, the disclosure of which is hereby incorporated by reference herein in its entirety.

### BACKGROUND OF THE INVENTION

Patients often require a total hip arthroplasty in the event of arthritis, instability, joint pain, femoral fracture, and the like that a patient may experience. A typical hip prosthesis used in a total hip arthroplasty includes a stem which functions as the base for proximal head replacement of the femur. Stems generally require a resection of the femur in preparation for implantation. With metaphyseal/diaphyseal engaging stems, a surgeon would typically resect a significant amount of good quality healthy bone stock to fit the stem into the femoral canal. Due to the loss of such healthy bone stock, press-fitted cement-less stems are inserted deeper into the canal than may be required otherwise in order to generate adequate fixation and stability. In this regard, conventional stems load the femur relatively distally, and therefore extend deep into the femoral canal to achieve sufficient initial stability and long-term fixation in the metaphyseal region. Consequently, the conventional stem often requires significant preparation of narrow femoral canals up to depths very often exceeding 100 millimeters. Such preparation typically involves extensive instrumentation, such as a family of sequentially increasing sizes of broaches, to prepare the femoral canal up to the required depth. Such instrumentation not only leads to excess equipment used in surgery, but may also lead to decreased stability due to repetitive insertion and removal of broaches of increasing size. Further, in order to be able to present such instruments to the femoral canal, the standard level of resection is distal of the femoral neck such that it leaves little to no bone remaining at the femoral neck. Therefore, further improvements are desirable.

### BRIEF SUMMARY OF THE INVENTION

The present disclosure describes a hip prosthesis having a curved, tapering stem including rasp teeth on a distal-lateral surface capable of self-broaching upon implantation. The stem may extend into the canal slightly past the level of the lesser trochanter, having a length relatively shorter than the conventional tapered wedge and other anatomic stems. The disclosed stem provides more anatomic loading on the medial calcar region of the proximal femur and promotes preservation of bone stock in the femoral neck during a total hip arthroplasty. The implant as described herein provides several benefits that improve the outcome of the procedure, such as a reduced moment arm, improved implant load distribution via greater anatomic loading on the implant, reduced potential for trochanteric fractures, greater head offset and increased joint stability, improved bone remodeling due to less bone resorption in the proximal femur, potential for reduced blood loss, greater preservation of soft tissue in the trochanteric region and potential to reduce thigh pain and improve the timing and experience of rehabilitation and recovery for the patient. Another potential benefit is that a greater amount of bone preserved in the neck may enable easier revisions for patients in the event of failure because there remains good bone stock proximal to the conventional resection level, which can subsequently be removed and revised with a conventional stem.

Preparation of the neck region of the femoral canal proximal to the level of the lesser trochanter may be performed robotically (e.g., with a rotary cutting tool such as a burr that removes cancellous bone) based on a CT-based 3D plan of the patient, which would enable initial stem insertion into the canal. The stem is intended to be self-broaching to simplify the surgical workflow. In other words, the stem itself can carve its own path in the bone and may not require initial shaping of the canal with other instruments, such as an associated family of sequentially increasing sizes of broaches. This may be achieved with the help of the proximal robotic preparation as well as one or more cutting rasp teeth features disposed on a relief surface on the distal-lateral portion of the stem, as will be discussed below in greater detail. For some patients, preparation of the neck region of the femoral canal may include the formation of a pilot hole using an instrument such as a curved awl, which is also discussed below in greater detail.

In certain embodiments, a femoral implant may include a spherical head positioned a proximal end of the implant. The femoral implant may further include a neck coupled to the head, the neck extending distally from the head. The femoral implant may further include a stem extending distally from the neck. The stem may have a medial side adapted to face a medial extent of the femur and a lateral side adapted to face a lateral extent of the femur when implanted therein. The medial side may have a concave curvature and the lateral side may have a convex curvature. The stem may include a plurality of teeth disposed on a distal portion of the lateral side of the stem such that the stem is configured to be self-broaching when implanted into the femur. The plurality of teeth may be arranged in a plurality of columns on the lateral side. The plurality of teeth may be arranged in an array forming straight rows and columns of teeth on the lateral side, the teeth spaced equally apart from one another. The plurality of teeth may be arranged on the lateral side in an array having a plurality of rows wherein the teeth of every other row are aligned in columns and the teeth of adjacent rows are offset.

The stem may include a flange between the distal end of the neck and the proximal end of the stem, the flange having a larger cross-section than the neck defining a peripheral lip. The proximal portion of the stem may include a porous structure. The porous structure may be positioned more proximally on the lateral side of the proximal stem portion than the medial side of the proximal stem portion. The medial side may have a transition point where the medial side changes from the first radius of curvature to the second radius of curvature, and the stem may define a transition plane between the porous structure and a non-porous portion of the stem, wherein the transition point may be located proximal to the transition plane. The stem may include an anterior side adapted to face an anterior extent of the femur and a posterior side adapted to face a posterior extent of the femur, and the stem may taper in both the medial-lateral direction and the anterior-posterior direction as the stem extends distally. The plurality of teeth may be disposed on a relief surface located on the lateral side of the stem. The relief surface may extend toward a central axis of the stem in a distal direction. The relief surface may intersect the convex curvature of the stem. The relief surface may be located at a relief portion of the stem. The relief portion of the stem may have a polygonal cross section. A distal portion of the stem may be polished. The concave curvature of the stem may extend from a proximal extent of the stem to a distal extent of the stem. The neck and stem may be monolithic, and the spherical head may be a modular piece coupleable to the neck and stem. The medial side of the stem may define a first radius of curvature on a proximal portion of the medial side of the stem. The medial side may further define a second radius of curvature on a distal portion of the medial side of the stem. The lateral side of the stem may define a third radius of curvature. The second and third radii of curvature may be different than the first radius of curvature.

In certain alternative embodiments, a femoral component may include a spherical head positioned at a proximal end of the implant. The femoral implant may further include a neck coupled to the head. The neck may extend distally from the head. The femoral implant may further include a stem extending distally from the neck. The stem may have a medial side adapted to face a medial extent of the femur and a lateral side adapted to face a lateral extent of the femur. The medial side may have a concave curvature and the lateral side may have a convex curvature. The concave curvature may be defined by a first and a second radius of curvature and the convex curvatures may be defined by a third radius of curvature. The stem may further include a relief surface intersecting the convex curvature and extending distally therefrom. The relief surface may have a plurality of teeth such that the stem is configured to be self-broaching when implanted into the femur.

In certain embodiments, a method of implanting a femoral prosthesis may include resecting a femur through a neck thereof between a head and greater trochanter of the femur such that a portion of the neck is preserved; impacting a femoral implant into the femur, the femoral implant including a stem having a porous bone-ingrowth portion and a convex lateral side that is defined by a radius of curvature and a plurality of teeth disposed on a distal portion of the lateral side; and cutting the bone using the plurality of teeth which is performed concurrently with the impacting step. The method may further include the step of planning the implantation using a CT-based 3D plan of a patient. The resecting step may include the use of a burr to resect the femur. The resecting step may be performed robotically. The method may further include creating a pilot hole that includes the use of a curved entry instrument to form the hole. The impacting step may include impacting the implant until the implant forms a press-fit with the resected portion of the femur. The impacting step may include impacting the implant until a flange disposed at the proximal end of the stem is on or within 2 millimeters of the resection level of the femur.

In certain alternative embodiments, a method of implanting a femoral implant may include planning procedure resection location on a femoral neck using a 3D CT scan of a patient; removing a proximal portion of the femoral neck using a robotically controlled high-speed burr so as to leave a distal portion of the femoral neck; preparing a pilot hole that extends into the femoral canal using a curved instrument; and impacting a femoral implant into the pilot hole, wherein the implant includes a plurality of teeth disposed on the distal-lateral surface of the implant to allow for self-broaching. The step of removing a proximal portion of the femoral neck may include robotically tracking the removal of the bone to ensure the bone removal accords with the surgical plan.

### BRIEF DESCRIPTION OF THE DRAWINGS

FIG. 1A is a perspective view of a hip implant according to an embodiment of the disclosure.

FIG. 1B is a schematic view of the hip implant of FIG. 1A illustrating radii of curvature thereof.

FIG. 1C is a close-up view of a distal portion of the hip implant of FIG. 1A.

FIG. 2 is a side view of the hip implant of FIG. 1A.

FIG. 3 is a front view of a solid model of a proximal portion of a femur.

FIG. 4A is a schematic cross-sectional view of a femur receiving the implant of FIG. 1A.

FIG. 4B is a close-up view of the distal portion of the implant in FIG. 4A disposed in the femur.

FIG. 5 is a schematic cross-sectional view of a femur with the implant of FIG. 1A fully implanted.

FIG. 6 is a front view of a hip stem implanted in a femur according to an embodiment of the disclosure.

FIG. 7 is a superior view of the hip of FIG. 1A implanted in the femur of FIG. 6.

FIG. 8 is a perspective view of a curved entry instrument according to an embodiment of the disclosure.

### DETAILED DESCRIPTION

The present disclosure will now be described more fully hereinafter with reference to the accompanying drawings, in which some, but not all aspects of the disclosure are shown. Indeed, the disclosure may be embodied in many different forms and should not be construed as being limited to the aspects set forth herein. Like numbers refer to like elements throughout.

As used herein, the term “proximal,” when used in connection with a device or components of a device, refers to the end of the device closer to the surgeon or user when the device is being used and implanted as intended. On the other hand, the term “distal,” when used in connection with a device or components of a device, refers to the end of the device farther away from the surgeon or user when the device is being used and implanted as intended. As used herein, the terms “substantially,” “generally,” “approximately,” and “about” are intended to mean that slight deviations from absolute are included within the scope of the term so modified.

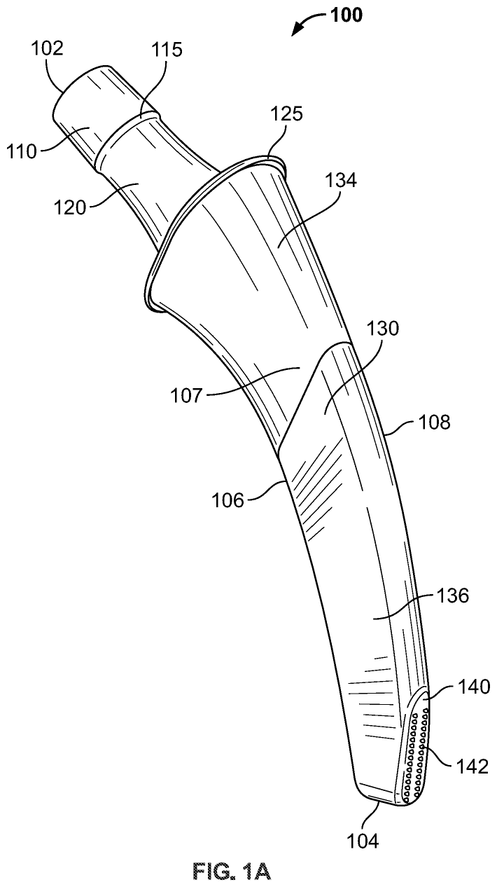

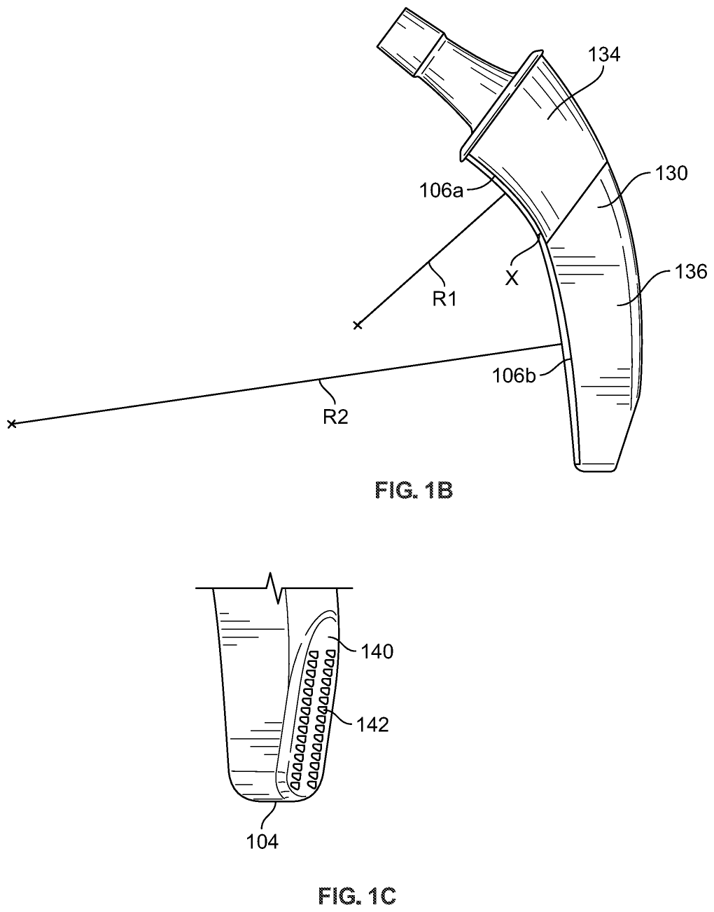

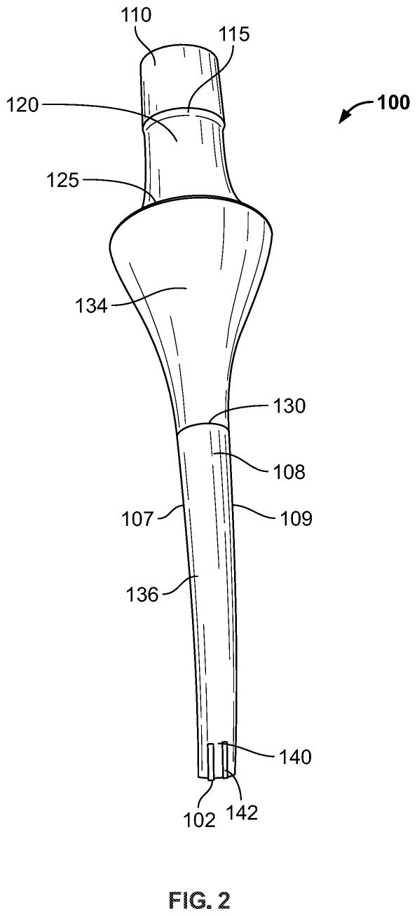

FIGS. 1-2 illustrate a hip implant 100 according to one embodiment of the disclosure. Implant 100 extends from a proximal end 102 to a distal end 104. Implant 100 may be implanted into a femur of a patient such that distal end 104 is the leading end (i.e., the first end to be inserted into the patient) whereas proximal end 102 is the trailing end that remains nearer the surgeon or user during implantation. A method of implantation will be discussed below in further detail.

At proximal end 102, implant 100 includes a head receiving portion 110, which is configured to receive a modular prosthetic spherical head (see FIG. 6). The spherical head may replace the anatomic femoral head and articulate with the acetabulum of the pelvis when the prosthesis is fully implanted. Head receiving portion 110 is generally frustoconical and therefore has a tapering diameter so as form a taper lock with a prosthetic head. Head receiving portion 110 has a first height. The distal end of head receiving portion 110 is coupled to a neck 120. Neck 120 may also be frustoconical and extends distally from head receiving portion 110. Neck 120 has a second height greater than the first height of head receiving portion 110 and a cross-sectional dimension less than the greatest cross-sectional dimension of head receiving portion 110, thus forming a peripheral lip 115 where head receiving portion 110 and neck 120 are coupled together. In some examples, the neck may have a height equal to or less than that of the head receiving portion. In still further examples, the head receiving portion may be rectangular, triangular, or any alternative shape, in which case the head may include a corresponding bore configured to fit to the shape of the head receiving portion. It is also contemplated that several parts of the implant may be modular, such as the head receiving portion, neck, proximal stem portion and/or distal stem portion.

A stem 130 is coupled to and extends distally from the distal end of neck 120. Stem 130 includes a proximal stem portion 134 and a distal stem portion 136. The proximal end of stem 130 includes a flange 125 where stem is coupled to neck 120. Flange 125 is generally stadium-shaped, i.e., flange 125 generally forms a rectangle with rounded corners. It is contemplated that the flange may define any shape that is suitable to prevent subsidence and does not substantially overhang the neck, such as an oval, rectangle, or the like. Flange 125 has a length and width which are each greater than the second diameter of neck 120, forming a peripheral shoulder at the connection between neck 120 and stem 130. When implant 100 is fully implanted, flange 125 sits at the neck resection level to provide initial stability and prevent subsidence of the stem after final seating. Stem 130 has a medial side 106 which, when the prosthesis is implanted as intended, will face toward a medial extent of the femur and the middle of the patient's body. Stem 130 further has a lateral side 108 which, when the prosthesis is implanted as intended, will face toward a lateral extent of the femur and the outside of the patient's body. Stem 130 further has an anterior side 107 which will face the front of the patient's body, and a posterior side 109 which will face the rear of a patient's body. It should be noted that the anterior and posterior sides are not limited to facing in their respective directions as defined above, but are merely defined this way for ease of illustration. Implant 100 may be implanted into a first femur on one side of the patient with the anterior side facing frontward and the posterior side facing rearward. However, implant 100 may be implanted into a second femur on the opposing side of the patient with the anterior side facing rearward and the posterior side facing frontward.

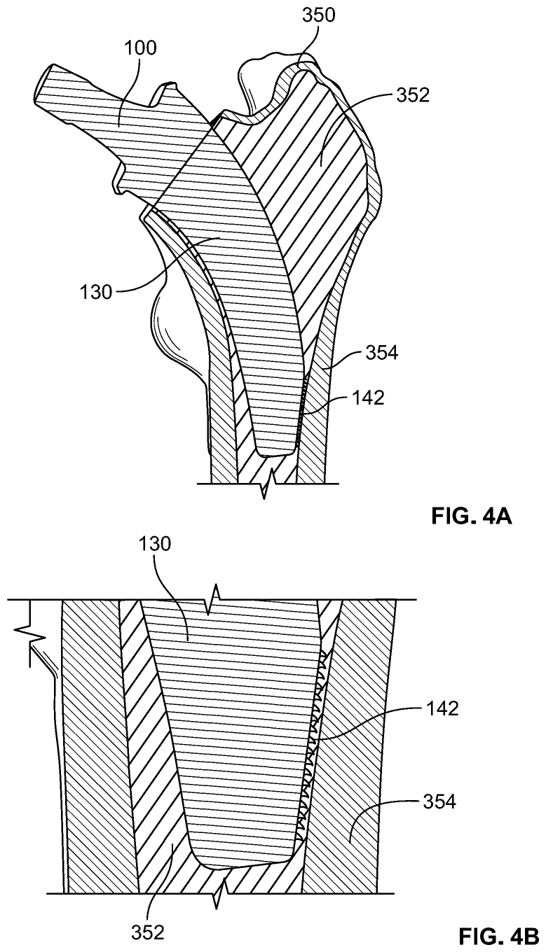

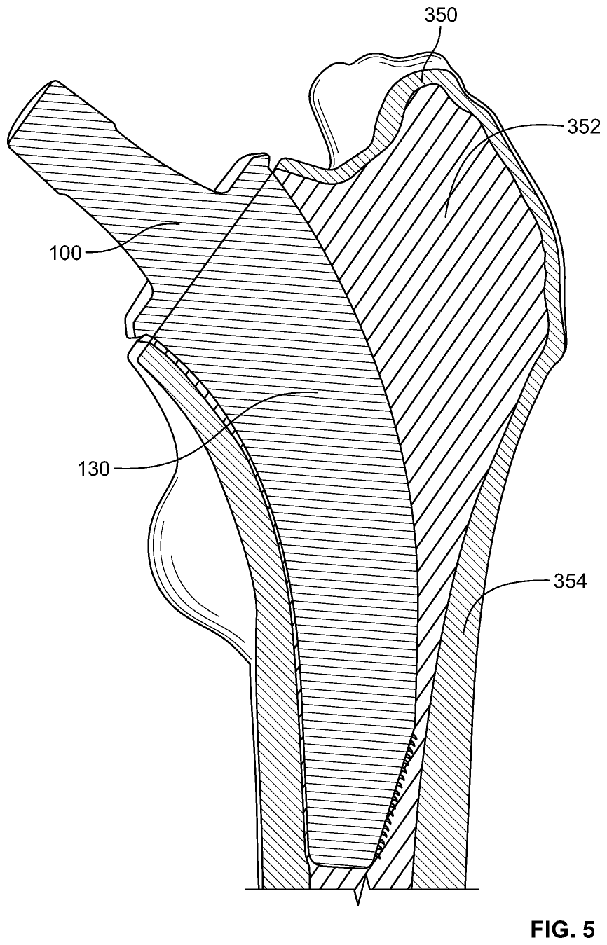

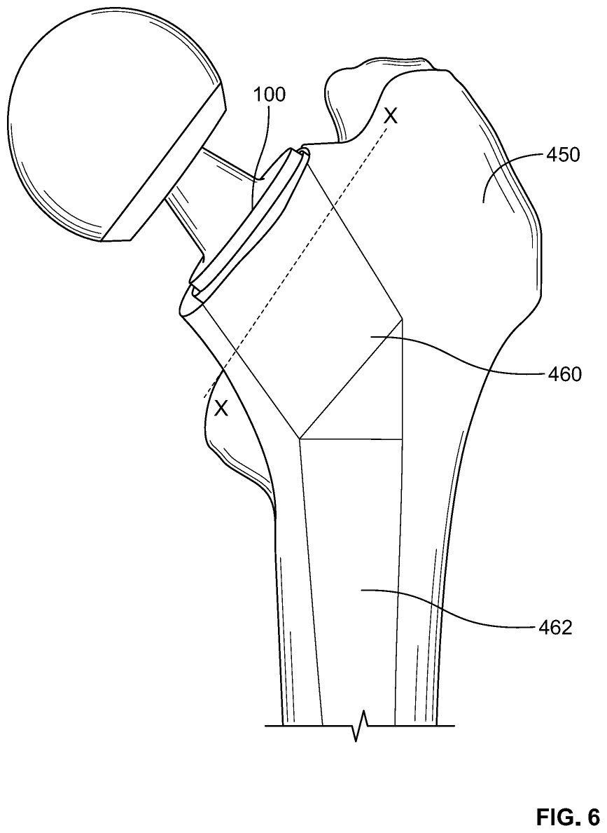

As stem 130 extends distally, stem 130 curves toward medial side 106. Thus, medial side 106 has a concave curvature which may transfer load to the proximal femoral neck and medial calcar region and may improve the press fit between stem 130 and the femoral neck. Lateral side 108 has a convex curvature, which may conserve bone at the femoral neck, improve press fit, and improve stem insertion by clearing the lateral cortices within the femoral shaft. As illustrated in FIG. 1B, proximal-medial side 106a of proximal stem portion 134 has a first radius of curvature R1 and distal-medial side 106b of distal stem portion 136 has a second radius of curvature R2 greater than the first radius of curvature R1. Proximal-medial side 106a having first radius of curvature R1 transitions to distal-medial side 106b having second radius of curvature R2 at a transition point X. Transition point X is defined as an intersection between the curved proximal medial surface 106a and curved distal medial surface 106b. As shown, the radius of curvature R2 is greater than radius of curvature R1. In this regard the center of curvature defined at the end of R2 is located further from stem medially than the center of curvature at the end of radius R1. However, as shown, radii R1 and R2 do not intersect. Such a geometry of the stem may help to conserve bone stock in the neck region and help with the insertion of stem 130 into bone so that stem 130 conforms to a patient's cortical bone at a medial aspect of a patient's femur as it is being inserted into an intramedullary canal thereof, as depicted in FIGS. 4A-5. Further, lateral side 108 of stem 130 has a third radius of curvature also greater than the first radius of curvature R1. The curvatures of the medial and lateral sides of the implant may be modified or customized to properly fit specific patients. The curvatures may be derived from the Stryker Orthopaedic Modeling and Analytics (SOMA) database.

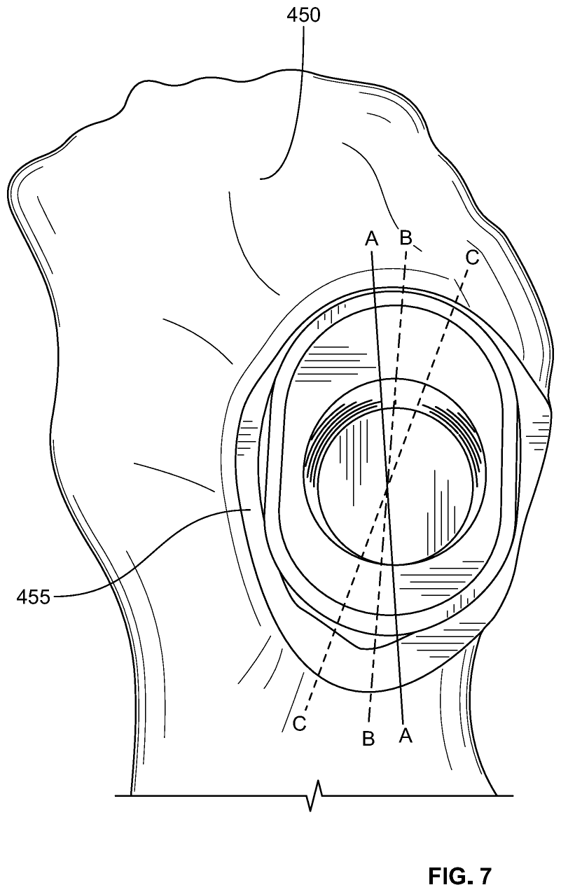

Proximal stem portion 134 may have a cross-sectional shape that is generally quadrilateral, such that the taper of proximal stem portion 134 clears all of the cortices of the femoral neck upon insertion except the general medial calcar region, which is approximately where proximal stem portion 134 is seated after insertion. As proximal stem portion 134 extends distally, the length and width of stem portion 134 taper, wherein the length is defined by the distance between medial side 106 and lateral side 108 and the width is defined by the distance between anterior side 107 and the posterior side 109. The anterior-posterior taper is illustrated more clearly in FIG. 2. The taper of stem 130 may help to prevent unintended distal fixation and fit patients with larger mismatches in version angle between the neck and proximal femur. In other words, the taper of the stem 130 may help to insert the stem 130 evenly into the canal and help prevent anteversion or retroversion of the stem 130 in the femoral neck relative to the proximal portion of the femur. The version angle of the stem 130 is best illustrated in FIG. 7, which shows the stem 130 evenly seated within the femoral neck 455 on all sides. The tapered profile of the stem 130 may clear the distal cortices of the proximal canal to allow the stem to be fully seated onto the medial calcar region. The distal surface of the flange 125 may contact or come substantially close to contacting the medial calcar region for the femur. In the embodiment of FIG. 7, the femoral neck 455, the stem 130 and the femoral canal each define a generally ovular shape, each having a major axis extending along a plane. The major axis of the femoral neck 455 is identified by axis A-A, the major axis of the stem 130 is identified by axis B-B, and the axis of the femoral canal is identified by axis C-C. As shown in FIG. 7, the major axis of each of the neck 455, stem 130 and canal extend along different planes. Thus, the tapered profile of the stem 130 may help to overcome the distinct shapes of the neck 455, stem 130 and canal shown by the varying major axes and may allow for a smooth and even insertion of the stem 130 into the femoral canal.

It is also contemplated that the entire stem 130 may taper in length and width. Proximal stem portion 134 includes a 3D porous coating to enable bone ingrowth and long-term fixation of implant 100 within the femur. A boundary between the porous coating and non-porous stem extends medially-laterally at an oblique angle relative to an axis of the stem such that the boundary is more proximal on the lateral side than on the medial side. Thus, the porous coating extends along a longer length on the medial side than on the lateral side. In this regard, the lateral side provides a longer smoother surface in order to provide clearance to enable the stem to fully seat within the canal, whereas the medial side provides greater fixation due to the additional porous surface area. The porous coating may be made from titanium or any biocompatible material such as hydroxyapatite or the like. Distal stem portion 136 is polished to avoid bony in-growth at the distal end of implant 100 and reduce stress shielding. Referring back to FIG. 1B, it should be noted that the transition point X between proximal-medial side 106a and distal-medial side 106b is located proximal to the boundary between the porous coating and the non-porous stem portion. In other words, the transition point X is located proximal to the distal-medial point of the proximal stem portion 134 having the porous coating.

As illustrated in FIG. 1A, distal stem portion 136 includes a relief surface 140 on lateral side 108. Relief surface 140 lies in a plane which extends in a distal direction toward a central axis of stem 130 and in a proximal direction to intersect the convex curvature of lateral side 108, forming a distal end of stem 130 tapering in length and having a polygonal cross-section. In other words, the concave curvature of medial side 106 may extend from flange 125 to a distal extent of stem 130, while the convex curvature of lateral side 108 may extend from flange 125 and terminate at relief surface 140. Relief surface 140 may then extend from the convex curvature to the distal extent of stem 130. Relief surface 140 may reduce lateral impingement on the inner surface of the femur and thereby ease insertion of implant 100. Relief surface 140 includes a plurality of teeth 142 disposed along and extending from the surface.

FIG. 1C illustrates a close-up view of relief surface 140 including teeth 142. Each tooth forms a sharp prong that extends in a direction perpendicular to relief surface 140. Teeth 142 are arranged in a grid comprising 18 rows and 3 columns. Teeth 142 may contact the lateral extent of the femur as implant 100 is being implanted, which will be discussed below in further detail with reference to the method of implantation. Teeth 142 form an abrasive surface to allow for advancement of the implant through the femoral canal. The combination of teeth 142 and relief surface 140 provide implant 100 the ability to self-broach during implantation to advance and seat itself within the femoral canal without the need for preparation of the canal with various broaches. In some examples, the relief surface may have a radius of curvature and the radius of curvature may be equal to the radius of curvature of the lateral side. In further examples, the teeth may be arranged on the relief surface in any number of rows and columns. In still further examples, the teeth may be arranged in an array having a plurality of rows with the teeth of every other row arranged in columns and the teeth of adjacent rows vertically offset. In still further examples, the teeth may be scattered around the relief surface in no particular arrangement positioned with varying distances between each tooth. Teeth may also be disposed on the anterior and/or posterior surfaces 107, 109 of the distal stem portion 136 such that teeth are positioned on up to three of the four surfaces of distal stem portion 136. Teeth may also be disposed on the distal tip 104 of the stem. It is contemplated that the teeth may be formed of any suitable geometry to optimize the self-broaching ability of the stem. For example, a tooth may extend straight from the surface of the stem or may have a radius of curvature. Further, a tooth may have any rake angle, break edge and may have any dimensions (e.g., length, width and depth of each tooth). A tooth may be formed of a solid material all the way through or may be hollowed out.

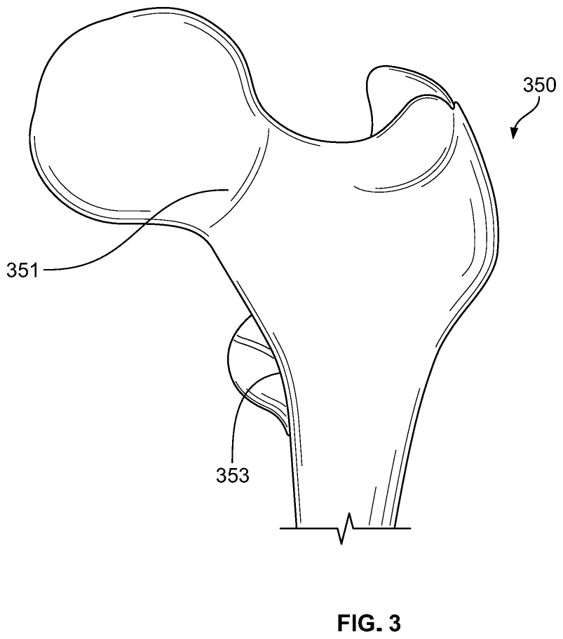

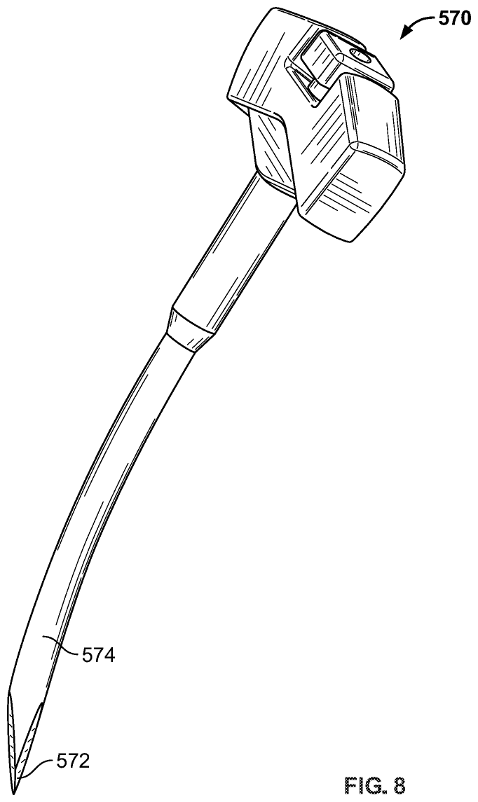

FIGS. 3-5 illustrate the resection, implantation and seating of implant 100 into a femur 350. Prior to surgery, the intended position of implant 100 may be planned using a CT-based plan of the patient. Femur 350 may be prepared robotically using a high-speed burr to resect the bone. Femur 350 may be resected through the neck 351 and the proximal canal up to the level of the lesser trochanter 353, as shown in FIG. 3. The femur may be resected such that a press fit connection may be formed between the femur and the intended fixation region of implant 100 when implanted into the femur. Robotic tracking of the burr may be employed while the burr is cutting to ensure the cut and the femur correspond to the pre-surgical plan. For some patients, e.g., patients with relatively smaller anatomy and/or tighter proximal canals, an instrument may be used to create a narrow pilot hole that extends into the femoral canal to prepare the canal to receive the distal portion of implant 100. The instrument used to form the pilot hole may be a curved awl, such as the instrument 570 shown in FIG. 8 having a sharp tip 572 and a curved stem 574 to form a curved hole that corresponds to the intended path of stem 130 during implantation. The instrument may be inserted past the medial boundary of the greater trochanteric canal space into the proximal femoral canal, thus creating a pilot hole through the cancellous bone of the proximal canal for the self-broaching stem. It is contemplated that the instrument may be controlled robotically or impacted manually. The instrument may be a single size that opens up a pilot hole up to a predetermined depth (e.g., lesser trochanter), which may be compatible with all sizes of the self-broaching stem. It should be noted that unlike a conventional broach, the curved awl instrument may not conform to the outer profile of the stem, and is only intended to ease entry of the stem into the distal canal. In this regard, implant 100 remains self-broaching even when a curved awl is utilized to form a pilot opening. The pilot hole may ease the insertion of stem 130 into the canal, but may be narrower than stem 130 such that a degree of force may be required during implantation and implant 100 may have a snug fit within the canal after implantation. Moreover, such curved entry instrument may not have teeth on it such that the bone within the femoral canal is not resected prior to the insertion of stem 130. After the pilot hole is formed, implant 100 may be impacted into the femur. Implant 100 widens the hole as stem 130 is inserted into the pilot hole resulting in a press fit relationship between stem 130 and the bone.

As shown in FIGS. 4A-B in which a substantial portion of stem 130 is implanted, teeth 142 disposed on relief surface 140 may be used to prepare the distal region of the femoral canal. As stem 130 is advanced distally through the cancellous bone 352 of femur 350, the distal portion of lateral side 108 may approach and contact the lateral extent of femoral cortical bone 354. This is at least due to the curved nature of stem 130 and the curved path the stem 130 takes as it is inserted into the bone. Teeth 142 and relief surface 140 allow for self-broaching of implant 100 by simultaneously preparing a pathway through the femoral canal for stem 130 while being advanced distally through the canal. FIG. 5 illustrates the final seated position of implant 100 within femur 350. Final seating of implant 100 may be achieved when flange 125 reaches the resection level, or a position that is within 2 millimeters of the resection level, and a desired press fit is achieved between the proximal portion of femur 350 and the corresponding portion of implant 100. In its final seating, medial side 106 of implant 100 abuts the medial extent of femur 350, and the length of stem 130 allows stem 130 to place its load on the medial calcar region of the proximal portion of femur 350, thus allowing for greater anatomic loading. It is also contemplated that the lateral side 108 of implant 100 may also abut a lateral extent of the femur.

It is contemplated that the pre-surgical plan of the patient may use any means of imaging, such as X-Ray, MRI, or the like, and the plan may also include 3D modeling of the implant and/or the femur of the patient through the use of CAD or similar software. It is also contemplated that any appropriate cutting means may be used to resect the femur, such as a reciprocating saw or the like. In some examples, the resection of the femur may be performed manually by a surgeon rather than robotically. It is contemplated that not all of the steps as described above are required for implantation. For example, a pilot hole may not be formed prior to implantation, but rather the implant may be impacted into the femur after the step of resecting.

FIGS. 6-7 illustrate implant 100 fully seated within a femur 450. As shown, a proximal section 460 of femur 450 that was robotically or manually resected is contrasted with a distal section 462 of femur 450 that was self-broached by the distal-lateral end of stem 130 during implantation. Additionally, axis X-X illustrates the level of resection of the femur in a conventional total hip arthroplasty in preparation for receiving an implant. Thus, the portion of femur 450 that remains intact proximal to axis X-X in FIG. 6 represents the amount of original bone that is preserved relative to a conventionally resected hip implant, thus providing each of the benefits as detailed above.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A femoral implant comprising:
a spherical head at a proximal end of the implant;
a neck extending distally from the head; and
a stem extending distally from the neck and having a medial side adapted to face a medial extent of the femur and a lateral side adapted to face a lateral extent of the femur when implanted therein, the medial side having a concave curvature, the lateral side having a convex curvature, the stem including a plurality of teeth disposed on a distal portion of the lateral side of the stem such that the stem is configured to be self-broaching when implanted into the femur.

2. The femoral implant of claim 1, wherein the stem includes a flange between the distal end of the neck and the proximal end of the stem, the flange having a larger cross-section than the neck defining a peripheral lip.

3. The femoral implant of claim 1, wherein the proximal portion of the stem includes a porous structure.

4. The femoral implant of claim 3, wherein the porous structure is positioned more proximally on the lateral side of the proximal stem portion than the medial side of the proximal stem portion.

5. The femoral implant of claim 3, wherein the medial side has a transition point where the medial side changes from the first radius of curvature to the second radius of curvature, and the stem defines a transition plane between the porous structure and a non-porous portion of the stem, wherein the transition point is located proximal to the transition plane.

6. The femoral implant of claim 1, wherein the stem includes an anterior side adapted to face an anterior extent of the femur and a posterior side adapted to face a posterior extent of the femur, the stem tapering in both the medial-lateral direction and the anterior-posterior direction as the stem extends distally.

7. The femoral implant of claim 1, wherein the plurality of teeth are disposed on a relief surface located on the lateral side of the stem, the relief surface extending toward a central axis of the stem in a distal direction, the relief surface intersecting the convex curvature of the stem.

8. The femoral implant of claim 7, wherein the relief surface is located at a relief portion of the stem, the relief portion of the stem having a polygonal cross section.

9. The femoral implant of claim 1, wherein the concave curvature of the stem extends from a proximal extent of the stem to a distal extent of the stem.

10. The femoral implant of claim 1, wherein the neck and stem are monolithic, and the spherical head is a modular piece coupleable to the neck and stem.

11. The femoral implant of claim 1, wherein the medial side of the stem defines a first radius of curvature on a proximal portion of the medial side of the stem, the medial side further defining a second radius of curvature on a distal portion of the medial side of the stem.

12. The femoral implant of claim 11, wherein the lateral side of the stem defines a third radius of curvature and the second and third radii of curvature are different than the first radius of curvature.

13. A femoral implant comprising:
a spherical head positioned at a proximal end of the implant;
a neck coupled to the head extending distally from the head;
a stem extending distally from the neck having a medial side adapted to face a medial extent of the femur and a lateral side adapted to face a lateral extent of the femur, the medial side having a concave curvature and the lateral side having a convex curvature, the concave curvature being defined by a first and a second radius of curvature and the convex curvatures being defined by a third radius of curvature, the stem further including a relief surface intersecting the convex curvature and extending distally therefrom, the relief surface having a plurality of teeth such that the stem is configured to be self-broaching when implanted into the femur.

14. A method of implanting a femoral prosthesis comprising:
resecting a femur through a neck thereof between a head and greater trochanter of the femur such that a portion of the neck is preserved;
impacting a femoral implant into the femur, the femoral implant including a stem having a porous bone-ingrowth portion and a convex lateral side that is defined by a radius of curvature and a plurality of teeth disposed on a distal portion of the lateral side; and
cutting the bone using the plurality of teeth which is performed concurrently with the impacting step.

15. The method of claim 14, further comprising the step of planning the implantation using a CT-based 3D plan of a patient.

16. The method of claim 14, wherein the resecting step includes the use of a burr to resect the femur.

17. The method of claim 14, wherein the resecting step is performed robotically.

18. The method of claim 14, further comprising creating a pilot hole that includes the use of a curved entry instrument to form the hole.

19. The method of claim 14, wherein the impacting step includes impacting the implant until the implant forms a press-fit with the resected portion of the femur.

20. The method of claim 14, wherein the impacting step includes impacting the implant until a flange disposed at the proximal end of the stem is on or within 2 millimeters of the resection level of the femur.
